(19) United States
(12) Patent Application Publication
(10) Pub. No.: US 2002/0161003 A1
(43) Pub. Date: Oct. 31, 2002

(54) Method and compositions for the eradication and control of methicillin-resistant staphylococcus aureus bacteria and the prevention of the development of antibiotic drug resistance in said bacteria

(76) Inventor: James C. Costin, Lower Gwynedd, PA (US)

Correspondence Address: Kenneth Watov, Watov & Kipnes, P.C., P.O. Box 247, Princeton Junction, NJ, 08550, US

(21) Appl. No.: 09/915,121

(22) Filed: Jul. 25, 2001

Related U.S. Application Data
(62) Division of application No. 09/265,640, filed on Mar. 10, 1999, now abandoned.
Application No. 09/265,640 is a continuation-in-part of application No. 09/151,885, filed on Sep. 11, 1998, now abandoned.

(51) Int. Cl. A61K 31/54

(52) U.S. Cl. 514/222.5

(57) ABSTRACT

The use of 4,4-methylenebis (tetrahydro-1,2,4-thiadiazine-1,2-dioxide) in the prevention and control of the development of antibiotic drug resistance in staphylococcus aureus bacteria and in the prevention of bacteria-to-bacteria transfer of genes capable of resisting antibiotics is disclosed.

This application is a continuation-in-part of U.S. Application Ser. No. 09/151,885 filed Sep. 11, 1998.

This invention relates to a method and compositions for the treatment of bacterial infection which reduces or eliminates the ability of bacteria to acquire resistance to antibiotic drug treatment. Moreover, this invention relates to a method and compositions for the reduction or elimination of bacteria-to-bacteria transfer of antibiotic drug resistance through genetic or other means.

Specifically, the present invention relates to the use of 4,4′-methylenebis(tetrahydro-1,2,4-thiadiazine-1,2-dioxide) known generically as taurolidine to treat antibiotic drug (e.g. gentamicin, methicillin and vancomycin) resistant bacterial infections, nosocomial infections and/or eradication of these organisms from an individual acting as a “carrier” for these organisms.

Further, the present invention relates to the use of taurolidine to prevent the development of antibiotic drug resistance in bacterial and nosocomial infections.

Lastly, the present invention relates to the use of taurolidine to prevent the bacteria-to-bacteria transfer of antibiotic drug resistance through genetic or other means.

The development of antimicrobial agents has, without question, been one of the crowning achievements of medical science in the latter half of the twentieth century. However, despite the fact that dozens of classes of compounds have been developed, microorganisms, especially bacteria, have developed resistance to virtually every agent which has been subjected to extensive clinical use. As we approach the end of the twentieth century, there has been a precipitous decline in the development of new antimicrobial agents. There are several reasons for this including the fact that most of the easy targets that allow selective toxicity for antimicrobial agents have been discovered and the fact that it is increasingly expensive to bring a new antimicrobial agent from discovery to the marketplace. There is, however, a major need for discovery of novel classes of antimicrobial agents to which multi-resistant bacteria remain susceptible. Taurolin is such a novel new antimicrobial agent. It has a formulation which comprises taurolidine (4˜- methylene bis (tetrahydro-1, 2, 4 thiadiazine 1, 1 dioxide). A derivative of aminosulphonic acid taurineamide, this is a novel bactericidal agent that has a unique spectrum of antimicrobial activity that, in preliminary tests, has included Gram-positive and Gram-negative bacteria and fungi. It has been subjected to early clinical trials and it appears to have useful activity in vivo when administered by intravenous or intraperitoneal routes. This compound also has the ability to neutralize endotoxin in vitro and it also exhibits marked anti-adherence properties in vitro.

Additionally, taurolidine has a low potential for toxicity. Doses of 600 mg/kg over 24 hours are non-toxic in experimental animals. Two percent (2%) solutions at doses of 100 mg/kg have been infused intravenously over 60 min in rabbits. The LD₅₀ in rats exceeds 4,000 mg/kg.

The emergence of multiple drug resistant enterococci and staphylococci currently poses an enormous threat, especially to hospitalized patients. Many such isolates are resistant to all currently used antimicrobials, and treatment with investigational agents is sometimes necessary. In addition, it has been shown in the laboratory that vancomycin resistance genes can be transferred to staphylococcus aureus in which they are expressed; therefore, there is great concern that this will occur in nature, jeopardizing the most effective current antimicrobial agent for treatment of infections due to methicillin-resistant strains of this species. Clearly, new agents with activities against enterococci and other resistant gram-positive pathogens are needed.

Taurolidine has demonstrated bactericidal activity against a broad range of microorganisms and taurolidine has the additional advantage of acting through mechanisms unlike those described for other currently available antimicrobial chemotherapeutic agents. This compound demonstrates activity in vitro against Enterococcus faecalis at concentrations <2000 μg/ml, with minimum inhibitory concentrations (MIC₅₀S) of 125-250 μg/ml and (MIC₉₀s) of 250-1000 μg/ml. The major metabolite, taurultam, is described as being approximately 50% as potent. Interestingly, taurolidine demonstrates antibacterial activity in vivo despite plasma concentrations which are an order of magnitude below the MIC. Reasons for this major discordance between in vitro and in vivo activities of the agent are not understood, but several factors may contribute. The compound demonstrates endotoxin neutralizing activity, inhibits adherence, is more active at low pH which may prevail at the site of infections or within phagolysosomes, is slightly more active when tested in serum- supplemented media, and inhibits potential bacterial toxins such as staphylococcal coagulase.

The precise mode of action of taurolidine has not been fully elucidated. In simple aqueous solution, taurolidine exists in equilibrium with taurultam and methylol-donating species. Current consensus suggests hydrolysis of taurolidine in vivo to methylol taurultam and taurultam in equilibrium. Upon liberation of one active N-methylol (hydroxymethyl) group from methylol taurultam, taurultam is further hydrolyzed via methylol taurineamide to taurine. Thus, three active N-methylol groups are liberated per molecule of taurolidine following reaction with bacterial or fungal cell constituents. These methylol groups have a high affinity for, and bind selectively and irreversibly to bacterial cell wall constituents to exert their bactericidal affect. Because of this unique mechanism of action, there is no reason to suspect cross-resistance with standard antimicrobial agents that do not share this mechanism of action.

Taurolidine's general characteristics include acceptable stability in the solid state when stored at ambient conditions, melting with decomposition at approximately 170° C. and the following solubility in aqueous solutions and organic solvents.

| | |
|---|---|
| Water | 1% at 20° C. |
| Dilute HCl | soluble |
| Dilute NaOH | soluble |
| CHCl₃ | insoluble |
| EtOH | sparingly soluble |
| DMF | 1 g in 2 mL/ca. 60° C. |
| Acetone | 1 g in 120 mL/Boiling |
| Ethanol | 1 g in 130 mL/Boiling |
| Methanol | 1 g in 170 mL/Boiling |
| Ethyl Acetate | 1 g in 200 mL/Boiling |

A saturated solution of taurolidine in deionized water has a pH of 7.4. The apparent partition coefficient of taurolidine between octanol and water (buffered at pH 7.2) is approximately 0.13 and would therefore not be predicted to accumulate to any significant extent in fatty tissues.

The synthesis of taurolidine is covered in a number of patents including U.S. Pat. No. 3,423,408; Switzerland No. 482,713 and United Kingdom No. 1,124,285 and is carried out in five stages:

Potassium phthalimidoethane sulphonate is prepared from taurinc, phthalic anhydride, glacial acetic acid and potassium acetate;

Potassium phthalimidoethane sulphonate is then converted to phthalimidoethane sulphonylchloride by chlorination with phosphorous oxychloride;

Phthalimidoethane sulphonylchloride is reacted with ammonia to form phthalimidoethane sulphonamide;

Phthalimidoethane sulphonylchloride is reacted with hydrazine hydrate and in the subsequent hydrazinolysis to form taurinamide hydrochloride; and

Taurolidine is prepared from taurinamide hydrochloride and formaldehyde.

The antimicrobial actions of taurolidine have been described in U.S. Pat. No. 3,423,408 and elsewhere in the literature. In addition, the following United States Patents describe various uses for and compositions containing taurolidine: U.S. Pat. No. 4,107,305, treatment of endotoxaemia; U.S. Pat. No. 4,337,251, elimination of adhesion formation as a result of surgery; U.S. Pat. No. 4,587,268, resorbable aqueous gels; U.S. Pat. No. 4,604,391, prevention of the occurrence of osteitis or osteomyelitis; U.S. Pat. No. 4,626,536, combatting toxic proteins or peptides in the blood; U.S. Pat. No. 4,772,468, treatment of bone cavities; and U.S. Pat. No. 4,882,149, directed to methods for filling congenital, surgical or traumatic defects with compositions comprising natural bone mineral having absorbed therein/thereon taurolidine.

Taurolidine has been shown to be safe and well tolerated at systemic doses exceeding 40 g/day and cumulative doses up to and exceeding 300 g.

The formulations of taurolidine generally utilized are sterile solutions containing 0.5%, 1.0% or 2.0% taurolidine for irrigation/lavage, wound instillation, intravenous or oral administration, primarily for the treatment or prevention of peritonitis, sepsis or osteitis/osteomyelitis. In addition, topical surgical gels containing 2.0% to about 4.0% are utilized for the treatment of osteitis/osteomyelitis.

It has long been the goal of the pharmaceutical industry to produce antibiotic medicinal substances that have the power to kill—or at least to arrest the growth of—many disease causing bacteria such as the streptococci, enterococci and staphylococci.

It has also been observed that the susceptibility of bacteria to various antibiotic medicines can change markedly over time, i.e., the antibiotic gentamicin was widely used for about ten years to treat staphylococcal infection until the bacteria acquired a resistance to gentamicin. The realization that infectious bacteria could become immune to all available antibiotics has raised alarm in the medical community which now cautions doctors that over prescribing antibiotics can hasten the evolution of resistant germs.

Moreover, a study by the Federal Centers for Disease Control in Atlanta, Georgia, has shown that nearly eight percent of all enterococci isolated in hospitals nationwide were resistant to vancomycin, the antibiotic considered to be the last line of defense against organisms impervious to other drugs. This was more than 20 times the rate of resistance to vancomycin detected only four years earlier.

Of equal concern to the medical community is the ability of a bacteria, once it has acquired an immunity to an antibiotic, to transfer such immunity to other bacterium.

Resistance to antibiotics is developed in bacterium cells in a small circle of DNA known as plasmid which is matter consisting of a double-stranded DNA that is apart from the chromosomes but carries genes for a variety of functions and can replicate itself. The genes are concerned with such functions as resistance to antibiotics. Plasmids are separate from the rest of the bacterium, and they can move quite easily from one bacterium to another. This transferability of plasmids enables resistance genes to spread rapidly even among different species of bacteria. Transfer between bacteria of plasmids is accomplished through the use of F pili which are fine filaments resembling flagellum which are outgrowths from the bacteria cells which normally function to propel the cell, however when the F pili attach to another cell, a bridge is formed which permits the plasmids to spread rapidly from one cell to another.

Antibiotics generally work by interfering with the construction of the bacterial cell wall. In the case of vancomycin-resistance, its action can be thwarted by the bacteria modifying the building blocks of their cell walls by substituting a molecule of lactic acid for one of analine. The most common plasmid that confers resistance to vancomycin has a package of nine genes that set in motion this modification. A first gene enables the bacterium to manufacture lactic acid, a second gene codes for an enzyme that can cleve the analine from the cell wall building block, the product of a third gene puts lactic acid in the analines place. Two more genes control the previous three ensuring that they are activated only in the presence of vancomycin. The remaining genes are involved in helping the resistance package mobilize itself in different ways.

It has now been found that taurolidine in addition to its known antimicrobial, antitoxin, antibacterial and antifungal properties destroys antibiotic resistant strains of staphylococci, enterococci and other bacterial and nosocomial infections, prevents the development of antibiotic resistance in staphylococci, enterococci and other bacterial and nosocomial infections and prevents the transfer of bacteria-to-bacteria drug resistance through genetic or other means.

Microbial adherence to mucosal epithelial cells is recognized as a significant step in the successful colonization of the intestinal, respiratory and genito-urinary tracts in the early stages of infection. The attachment and agglomeration of organisms is important, both in the pathogenesis of infection and in limiting the response to antibiotic treatment.

Taurolidine has been found to significantly reduce the adherence of buccal and vaginal isolates of candida albicans blastospores and urine isolates of escherichia coli and staphylococcus saprophyticus to epithelial cells. Light microscopy and radio-isotopic counting methods were used to quantify the adherence of the microorganisms to either uropithelial or buccal epithelial cells.

Treatment of either epithelial cells or microorganisms with taurolidine resulted in reduced adherence of microorganisms.

Using a thirty minute contact time, a range of taurolidine concentrations on the order of 0.05% to about 2.0% w/v were examined for antiadherence activity. Significant decreases in candida blastospore adherence were observed at concentrations of less than 0.1% w/v taurolidine. Maximum reductions in adherence, on the order of about 65% of control were observed when concentrations of taurolidine greater than about 0.5% w/v. Increasing the taurolidine concentration beyond this level did not produce a concomitant increase in antiadherence activity. Conversely, dilution of taurolidine concentration may proceed to a considerable extent before its capacity for antiadherence is lost.

The foregoing demonstrates that taurolidine exerts an antiadherence activity via a chemical modification of outer surface structures such as fimbriae causing agglutination or disappearance of the structures. The effect of taurolidine on these structures which contribute to the initiation of infection and in determining the pathogenicity of the organism is clear evidence of one aspect of taurolidine's mechanism of action in preventing infection or reducing its severity.

As noted above, taurolidine's mechanism of action unlike that of known antibiotics is based on a chemical reaction. While not being bound by any theory, during the metabolism of taurolidine to taurinamide and ultimately taurine and water, methylol groups are liberated which chemically react with the mureins in the bacterial cell walls this results in the denaturing of the complex polysaccharide and liposaccharide components of the bacterial cell wall as well as changing the double stranded DNA of the plasmid to a denatured or single stranded DNA.

Example 1, which follows, demonstrates taurolidine's activity against vancomycin-resistant enterococci.

### EXAMPLE 1

Twelve clinical isolates of vancomycin-resistant enterococci, each with a vancomycin minimum inhibitory concentration of <128 μg/ml were challenged in vitro with taurolidine by pulsed-field gel electrophoresis. Ten of the vancomycin-resistant enterococci strains were genotypically distinct and two of the strains were genetically-related.

Additionally, taurolidine activity was tested against vancomycin-sensitive enterococci-E.Faecalis (American Type Culture Collection #29212) and E.Faecium (American Type Culture Collection #35667). Susceptibility testing was performed using broth dilution methods and geometric means were used to determine the minimum inhibitory concentration and minimum bactericidal concentration values.

For the twelve vancomycin-resistant enterococci strains:

$${\underline{MIC}}_{50} = \underline{400} µg \text{/} ml$$

$${MIC}_{90} = 500 µg \text{/} ml$$

$${\underline{MBC}}_{50} = \underline{1010} µg \text{/} ml$$

$${MBC}_{90} = 1500 µg \text{/} ml$$

For the American Type Culture Collection strains of E.Faecalis and E.Faecium:

$$\begin{matrix}\underline{MIC} = \underline{310} µg \text{/} ml \\ MBC = 960 µg \text{/} ml\end{matrix} and \begin{matrix}\underline{360} µg \text{/} ml \\ 980 µg \text{/} ml ,\end{matrix} respectively .$$

For two vancomycin-resistant strains, the minimum inhibitory concentration:

at pH 5=100 μg/ml

at pH 6=200 μg/ml

at pH 6.5=500 μg/ml

at pH 7=500 μg/ml

MIC at pH 5=50 μg/ml

MIC at pH 6=200-500 μg/ml and

MIC at pH 7=500 μg/ml

For two methicillin-resistant staphylococcus aureus isolates, the minimum inhibitory concentration with no serum added equals 500-1000 μg/ml and the minimum bactericidal concentration equals 1000-2000 μg/ml. After adding 95% rabbit serum, the minimum inhibitory concentration equals 500 μg/ml and the minimum bactericidal concentration equals 500-1000 μg/ml.

The foregoing data demonstrates that taurolidine has inhibitory and cidal activity against vancomycin-intermediate susceptibility staphylococcus aureus isolates and against methicillin-resistant staphylococcus aureus isolates.

The addition of serum did not have any significant effect on the activity of taurolidine. The minimum inhibitory concentration and the minimum bactericidal concentration at physiologic pH are above achievable serum levels, however, the data clearly demonstrates that taurolidine does have an effect on nasopharyngeal vancomycin-intermediate susceptibility staphylococcus aureus and methicillin-resistant staphylococcus aureus carriage if applied topically.

Thus, the medical community has a need for a protective agent against methicillin-resistant staphylococcus aureus which can be readily utilized in pharmaceutical formulations. The present invention fulfills that need.

In general, the compositions of the present invention can be readily utilized in pharmaceutical and/or nutritional formulations. The disclosed medicament may be used alone or in combination with other known antimicrobial agents such as gentamicin, methicillin and vancomycin together with a pharmacologically acceptable carrier and may be in capsule, tablet, powder, suspension or liquid form.

The formulations of taurolidine generally utilized are sterile solutions containing about 0.5%, 1.0%, 2.0% or about 4.0% taurolidine.

Solid carriers and diluents suitable for use include sugars such as lactose and sucrose, cellulose derivatives such as carboxymethyl cellulose, ethylcellulose, methylcellulose, etc., gelatin including hard and soft gelatin capsules, talc, cornstarch, stearic acid and magnesium stearate.

Suspension formulations may additionally contain benzoic acid, coloring, natural and artificial flavors, glycerin, kaolin, magnesium, aluminum silicate, methyl paraben, pectin, purified water, saccharin, sodium hydroxide and sucrose.

The percentage of taurolidine in the composition can be varied over wide limits and the quantity of medicament furnished by each individual tablet, capsule, solution or suspension is relatively unimportant since the indicated daily dose can be reached by administering either one or a plurality of capsules, tablets or suspensions.

The following non-limiting Examples are provided to illustrate further the present invention:

### EXAMPLE 3

### Solution

Taurolidine: 400 g

Polyvinylpyrrolidone: 100 g

Sterile Water to: 20 liters

15 Liters double distilled pyrogen free water are filled into a 25 liter glass vessel with stirrer and intensive reflux device and heated to 50° C. with stirring. The taurolidine 400 g is added followed by polyvinylpyrrolidone 1000 g. After dissolution, the solution is cooled and the pH adjusted to 6.0 with a few drops of 0.1N hydrochloric acid. The solution is then passed through an adsorption filter to remove microorganisms and pyrogens and through a sterilising millipore filter before being filled into 100 ml vials which are finally autoclaved.

### EXAMPLE 4

### Solution

Taurolidine: 990 g

Sterile Water ad: 22 liters

The taurolidine is dissolved in the sterile water and filled into sterile boffles, 250 ml in each.

### EXAMPLE 5

### Tablet

Taurolidine: 500 g

Amylum maydis: 60 g

Kollidone 25: 50 g (polyvinylpyrrolidone)

Plasdon XL: 20 g

Magnesium stearate: 6 g

Distilled water: 200 g

1000 tablets, each containing 500 mg taurolidine, are produced by conventional means using the above formulation.

In an alternative tablet formulation, the amylum maydis is replaced by 60 g amylum orizae.

### EXAMPLE 6

### Solution

Taurolidine: 440 g

Pharmaceutical gelatin: 88 g

Sodium chloride: 99 g

Sterile water to: 22 liters

The components are dissolved in the sterile water, if necessary using gentle warming and sonication. The solution is then filled into sterile bottles, 500 ml in each.

The foregoing description is given for clearness of understanding of the invention only, and no unnecessary limitations should be understood therefrom, as modifications will be obvious to those skilled in the art.

Various modifications may be made in the invention described herein. Accordingly, the scope of the invention is defined in the following claims wherein:

What is claimed is:

1. A method for the eradication and control of methicillin-resistant staphylococcus aureus and for the prevention of the development of antibiotic drug resistance in staphylococcus aureus bacteria which comprises administering to a human or other warm blooded animal, infected with such bacteria, the compound 4,4-methylenebis(tetrahydro-1,2,4-thiadiazine-1,2-dioxide).

2. A method for the prevention of bacteria to bacteria transfer of plasmid materials containing genes capable of resisting antibiotics which comprises administering to a human or other warm blooded animal harboring bacteria containing such plasmids the compound 4,4-methylenebis (tetrahydro-1,2,4-thiadiazine-1,2-dioxide).

3. The method of claim 1 wherein said 4,4-methylenebis (tetrahydro-1,2,4-thiadiazine-1,2-dioxide) is combined with an additional antibiotic or antibiotics.
